(19) United States
(12) Patent Application Publication
(10) Pub. No.: US 2024/0294458 A1
(43) Pub. Date: Sep. 5, 2024

(54) CATALYTIC HYDROGENATION OF AROMATIC NITRO COMPOUNDS

(71) Applicant: Hoffmann-La Roche Inc., Little Falls, NJ (US)

(72) Inventor: Paolo Tosatti, Binningen (CH)

(73) Assignee: Hoffmann-La Roche Inc., Little Falls, NJ (US)

(21) Appl. No.: 18/657,765

(22) Filed: May 7, 2024

Related U.S. Application Data
(63) Continuation of application No. PCT/EP2022/080899, filed on Nov. 7, 2022.

(30) Foreign Application Priority Data
Nov. 8, 2021 (EP) 21206827.4

(51) Int. Cl. C07C 209/36

(52) U.S. Cl. CPC C07C 209/36; C07C 2523/44; C07C 2527/20

(57) ABSTRACT

The invention provides a process for manufacturing an aniline 2, wherein PG denotes hydrogen or an amino protective group.

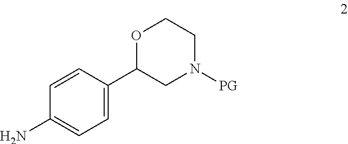

that is suitable for large-scale manufacturing of said aniline 2.

### CROSS-REFERENCE TO RELATED APPLICATIONS

This application is a continuation of International Application No. PCT/EP2022/080899 having an international filing date of Nov. 7, 2022, which claims benefit of priority to European Patent Application No. 21206827.4, filed Nov. 8, 2021, each of which is incorporated herein by reference in its entirety

### FIELD OF THE INVENTION

The invention relates to a novel process for manufacturing an aniline 2,

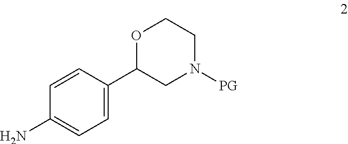

wherein PG denotes hydrogen or an amino protective group. The process according to the invention is particularly suitable for large-scale manufacturing of aniline 2 under GMP conditions.

### BACKGROUND OF THE INVENTION

Anilines 2 are crucial intermetidates in the synthesis of ralmitaront (Formula IV), a partial agonist of the TAAR1 (PCT application WO2017157873).

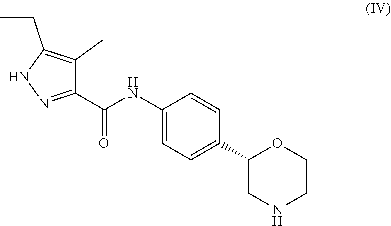 [SMILES: CCc1[nH]nc(C(=O)Nc2ccc([C@H]3CNCCO3)cc2)c1C]

For marketing products, it is necessary to produce pharmaceuticals in large quantities and according to good manufacturing practice (“GMP”). Hence, high-yielding, cheap, safe and reproducible syntheses are of utmost importance.

WO2015086495 discloses a process for making anilines 2, which involves catalytic hydrogenation of nitroarenes 1 in protic solvents.

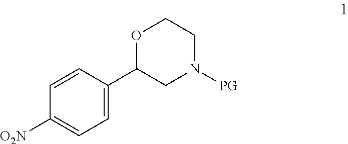

However, it has now been found that using a protic solvent for the hydrogenation, as described in WO2015086495, hampers the subsequent workup and isolation procedure on an industrial scale. Namely, on an industrial scale, it is preferable to crystallize and filter said anilines 2 after the hydrogenation step. The crystallization of anilines 2 has been found to require aprotic solvent systems, such as a mixture of TBME and heptane. Accordingly, if the hydrogenation of nitroarene 1 is performed in a protic solvent, such as methanol, a solvent swap is required for the subsequent crystallization step. A solvent swap generally consumes time and energy (distillation of large amounts of solvent), among other drawbacks.

What is more, the use of protic solvents in the hydrogenation of nitroarenes 1 has been found to produce varying trace amounts of side products, which is highly problematic when working under GMP conditions. Thus, for example, when ethanol was used as a solvent for the catalytic hydrogenation of (S)-tert-butyl 2-(4-nitrophenyl)morpholine-4-carboxylate (II),

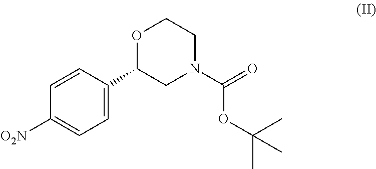 [SMILES: CC(C)(C)OC(=O)N1CCO[C@@H](c2ccc([N+](=O)[O-])cc2)C1]

(S)-tert-butyl 2-(4-ethylamino)morpholine-4-carboxylate (III) was observed as a side product, among others.

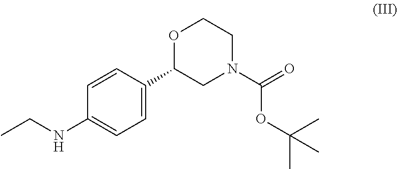 [SMILES: CCNc1ccc([C@H]2CN(C(=O)OC(C)(C)C)CCO2)cc1]

Therefore, there is a need for a new process for manufacturing anilines 2.

### SUMMARY OF THE INVENTION

It has now been found that nitroarenes 1 can be hydrogenated in an aprotic solvent, greatly facilitating the workup procedure of the resulting anilines 2 on an industrial scale. In addition, the hydrogenation, when performed in an aprotic solvent, surprisingly does not lead to the formation of any side product.

Thus, in a first aspect, the present invention provides a process for manufacturing an aniline 2, wherein PG denotes an amino protective group

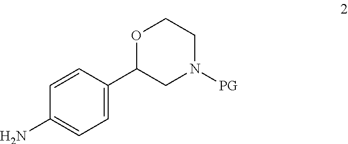

comprising:
reacting a nitroarene 1, wherein PG denotes an amino protective group, with hydrogen

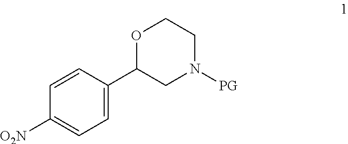

(i) in the presence of a transition metal catalyst;
(ii) in an aprotic solvent;
   to form said aniline 2.

In a further aspect, the present invention provides an aniline 2, wherein PG denotes an amino protective group

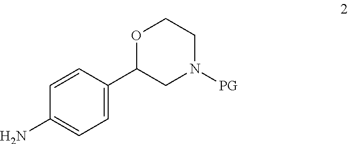

when manufactured according to the inventive process described herein.

In a further aspect, the present invention provides a process for manufacturing 5-ethyl-4-methyl-N-[4-[(2S) morpholin-2-yl]phenyl]-1H-pyrazole-3-carboxamide (Formula IV), or a pharmaceutically acceptable salt thereof,

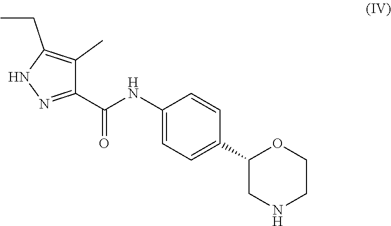 [SMILES: CCc1[nH]nc(C(=O)Nc2ccc([C@H]3CNCCO3)cc2)c1C]

comprising the inventive process described herein.

In a further aspect, the present invention provides the use of the process according to the invention for the manufacture of 5-ethyl-4-methyl-N-[4-[(2S) morpholin-2-yl]phenyl]-1H-pyrazole-3-carboxamide (Formula IV), or a pharmaceutically acceptable salt thereof.

### BRIEF DESCRIPTION OF THE FIGURES

FIG. 1 shows the conversion of (S)-tert-butyl 2-(4-nitrophenyl)morpholine-4-carboxylate into (S)-tert-butyl 2-(4-aminophenyl)morpholine-4-carboxylate over 8 h as a function of time for the two experiments described as Examples 1 and 2 herein. Conversion is determined as the amount of hydrogen consumed during the course of the reaction. In the case where no water additive was used, the temperature was increased by 20° C. to still achieve full conversion within ca. 6 h, as indicated in the FIGURE.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims and the abstract), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims and the abstract), or to any novel one, or any novel combination, of the steps of any method or process so disclosed.

The term “elevated pressure” refers to any pressure above ambient (i.e., atmospheric) pressure.

The term “elevated temperature” refers to any temperature above ambient (i.e., room) temperature.

The term “catalyst loading” refers to the amound of catalyst relative to a given reactant, calculated in weight percent (“% wt/wt”). In cases where a catalyst is provided in a wet form, e.g. wet Pd/C, the catalyst loading is calculated based on the amount of dry catalyst.

The term “protective group” (PG) denotes a group which selectively blocks a reactive site in a multifunctional compound such that a chemical reaction can be carried out selectively at another unprotected reactive site in the meaning conventionally associated with it in synthetic chemistry. Protective groups can be removed at the appropriate point. Exemplary amino protective groups are Boc (tert-butoxycarbonyl), benzyl, 4-methoxybenzyl, benzhydryl, Fmoc (fluorenylmethoxycarbonyl), Cbz (benzyloxycarbonyl), Moz (p-methoxybenzyl carbonyl), Troc (2,2,2-trichloroethoxycarbonyl), Teoc (2-(Trimethylsilyl)ethoxycarbonyl), Adoc (adamantoxycarbonyl), formyl, acetyl, and cyclobutoxycarbonyl. Further particular amino protective groups are tert-butoxycarbonyl (Boc) and fluorenylmethoxycarbonyl (Fmoc). A more particular protecting group is tert-butoxycarbonyl (Boc). Exemplary protecting groups and their application in organic synthesis are described, for example, in “Protective Groups in Organic Chemistry” by T. W. Greene and P. G. M. Wutts, 5th Ed., 2014, John Wiley & Sons, N.Y, which is included herein by reference in its entirety.

### Manufacturing Process

In a first aspect, the present invention provides a process for manufacturing an aniline 2, wherein PG denotes an amino protective group

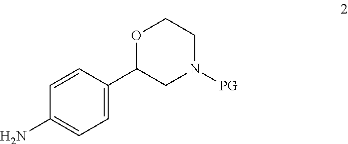

comprising:
reacting a nitroarene 1, wherein PG denotes an amino protective group, with hydrogen

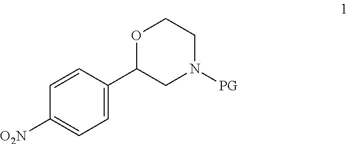

(i) in the presence of a transition metal catalyst;
(ii) in an aprotic solvent;
   to form said aniline 2.

In one embodiment, the present invention provides a process for manufacturing an aniline 2, wherein PG denotes hydrogen or an amino protective group

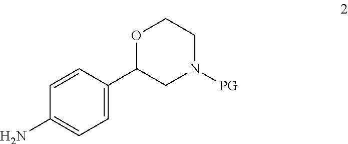

comprising:
reacting a nitroarene 1, wherein PG denotes an amino protective group, with hydrogen

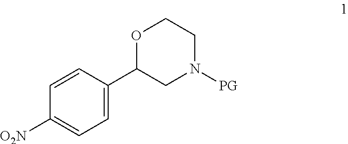

(i) in the presence of a transition metal catalyst;
(ii) in an aprotic solvent;
   to form said aniline 2.

In one embodiment, each amino protective group is independently selected from Boc (t-butoxycarbonyl), benzyl, 4-methoxybenzyl, benzhydryl, Fmoc (fluorenylmethoxycarbonyl), Cbz (benzyloxycarbonyl), Moz (p-methoxybenzyl carbonyl), Troc (2,2,2-trichloroethoxycarbonyl), Teoc (2-(Trimethylsilyl)ethoxycarbonyl), Adoc (adamantoxycarbonyl), formyl, acetyl, and cyclobutoxycarbonyl.

In a preferred embodiment, the amino protective group is Boc (t-butoxycarbonyl).

In one embodiment, said nitroarene 1 is nitroarene 1a, and said aniline 2 is aniline 2a

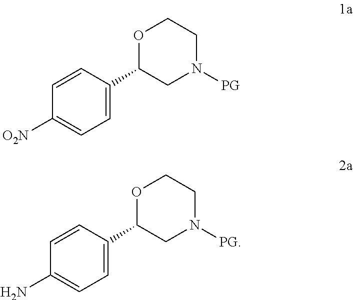

In one embodiment, said nitroarene 1 is nitroarene 1b, and said aniline 2 is aniline 2b

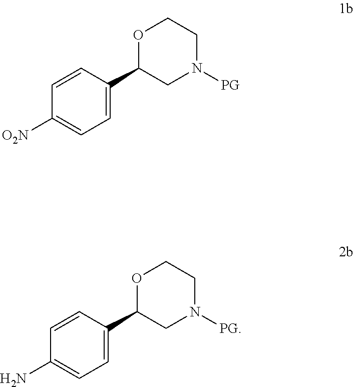

In a preferred embodiment, said nitroarene 1 is (S)-tert-butyl 2-(4-nitrophenyl)morpholine-4-carboxylate (II)

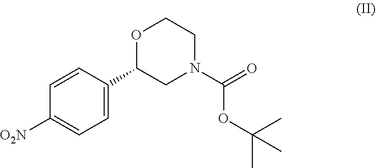 [SMILES: CC(C)(C)OC(=O)N1CCO[C@@H](c2ccc([N+](=O)[O-])cc2)C1]

In a preferred embodiment, said aniline 2 is (S)-tert-butyl 2-(4-aminophenyl)morpholine-4-carboxylate (I)

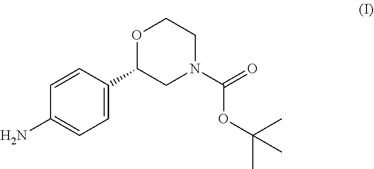 [SMILES: CC(C)(C)OC(=O)N1CCO[C@@H](c2ccc(N)cc2)C1]

In one embodiment, said transition metal catalyst is selected from Pt, Pd, Pt—V and Ni, wherein each of said Pt, Pd, Pt—V and Ni is on a solid support. In one embodiment, said solid support is selected from activated carbon, allumina, silica and an aluminium alloy.

In one embodiment, said transition metal catalyst is selected from PtO₂, Pd/C, Pt—V/C, Pt/C, and Raney Ni.

In a preferred embodiment, said transition metal catalyst is Pd/C.

In a particularly preferred embodiment, said transition metal catalyst is Pd/C and contains 5% wt/wt of palladium relative to charcoal (5% Pd/C).

In a further particularly preferred embodiment, said transition metal catalyst is Evonik Noblyst® P1093 5% Pd/C.

In one embodiment, the catalyst loading is 0.1% wt/wt to 1% wt/wt relative to nitroarene 1.

In a preferred embodiment, the catalyst loading is 0.4% wt/wt to 0.6% wt/wt relative to nitroarene 1.

In a particularly preferred embodiment, the catalyst loading is 0.5% wt/wt relative to nitroarene 1.

In a further particularly preferred embodiment, the catalyst loading is 0.55% wt/wt relative to nitroarene 1.

In one embodiment, said aprotic solvent is an ether.

In a preferred embodiment, said ether is tert-butyl methyl ether (TBME).

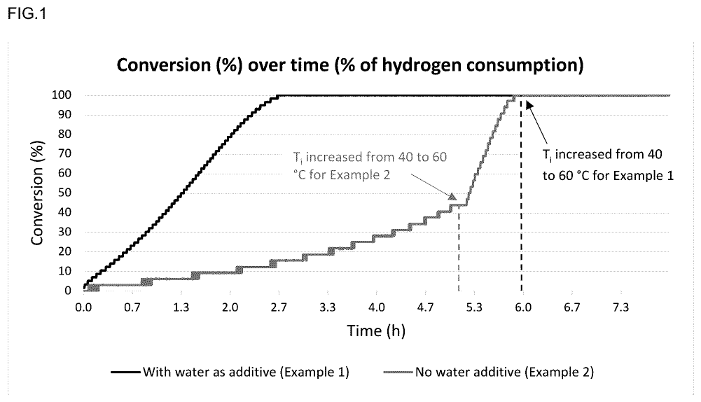

While using an aprotic solvent for the hydrogenation of nitroarene 1 to form aniline 2 solved the problems observed with the process described in WO2015086495, the reaction was found to be slow and require elevated reaction temperatures. Surprisingly, adding trace amounts of water to the reaction mixture resulted in a dramatically increased conversion rate of nitroarene 1 to aniline 2 (FIG. 1). In addition, lower temperatures were required to achieve full conversion.

Thus, in one embodiment, said aprotic solvent contains trace amounts of water.

In one embodiment, said trace amounts of water are 0.01% wt/wt to 0.1% wt/wt relative to the aprotic solvent.

In a preferred embodiment, said trace amounts of water are 0.05% wt/wt to 0.5% wt/wt relative to the aprotic solvent.

In a particularly preferred embodiment, said trace amounts of water are 0.25% wt/wt relative to the aprotic solvent.

In one embodiment, the process of the invention is conducted at elevated temperature.

In one embodiment, said elevated temperature is 35° C. to the boiling point of the reaction mixture.

In a preferred embodiment, said elevated temperature is 40° C. to 60° C.

In one embodiment, the process of the invention is conducted at elevated hydrogen pressure.

In one embodiment, said elevated hydrogen pressure is 1 barg to 10 barg.

In a preferred embodiment, said elevated hydrogen pressure is 3 barg.

In one embodiment:
(i) the transition metal catalyst is selected from Pt, Pd, Pt—V and Ni, wherein each of said Pt, Pd, Pt—V and Ni is on a solid support, preferably wherein said transition metal catalyst is selected from PtO₂, Pd/C, Pt—V/C, Pt/C, and Raney Ni;
(ii) the catalyst loading is 0.1% wt/wt to 1% wt/wt relative to nitroarene 1;
(iii) the aprotic solvent is an ether;
(iv) the aprotic solvent contains 0.01% wt/wt to 0.1% wt/wt of water relative to the aprotic solvent;
(v) the process is conducted between 35° C. and the boiling point of the reaction mixture; and
(vi) at a hydrogen pressure of 1 barg to 10 barg.

In a preferred embodiment:
(i) the transition metal catalyst is Pd/C;
(ii) the catalyst loading is 0.4% wt/wt to 0.6% wt/wt, in particular 0.55% wt/wt relative to nitroarene 1;
(iii) the aprotic solvent is tert-butyl methyl ether (TBME);
(iv) the aprotic solvent contains 0.05% wt/wt to 0.5% wt/wt of water relative to the aprotic solvent;
(v) the process is conducted at 40° C. to 60° C.; and
(vi) at a hydrogen pressure of 3 barg.

In a particularly preferred embodiment, the process according to the invention is:

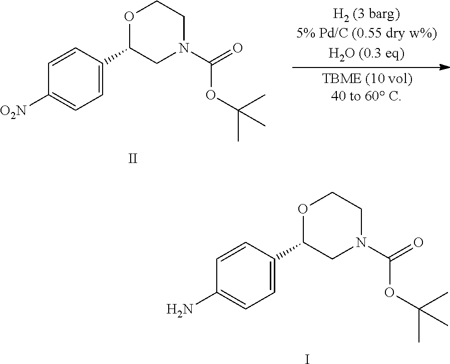

In one aspect, the present invention provides an aniline 2, wherein PG denotes hydrogen or an amino protective group,

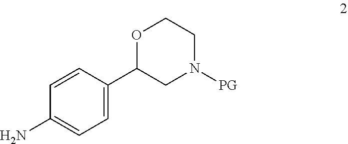

when manufactured according to the process of the invention.

In one aspect, the present invention provides an aniline 2, wherein PG denotes an amino protective group,

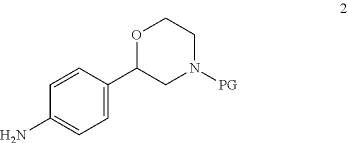

when manufactured according to the process of the invention.

In a further aspect, the present invention provides a process for manufacturing 5-ethyl-4-methyl-N-[4-[(2S) morpholin-2-yl]phenyl]-1H-pyrazole-3-carboxamide (Formula IV), or a pharmaceutically acceptable salt thereof, comprising the process according to the invention.

In a further aspect, the present invention provides the use of the process according to the invention for the manufacture of 5-ethyl-4-methyl-N-[4-[(2S) morpholin-2-yl]phenyl]-1H-pyrazole-3-carboxamide (Formula IV), or a pharmaceutically acceptable salt thereof.

### EXAMPLES

The invention will be more fully understood by reference to the following examples. The claims should not, however, be construed as limited to the scope of the examples.

The following abbreviations are used in the present text:
TBME tert-butyl methyl ether
Tᵢ internal temperature
barg bar gauge

### Example 1

### Preparation of (S)-tert-butyl 2-(4-aminophenyl)morpholine-4-carboxylate (I) in a Solvent Containing Trace Amounts of Water

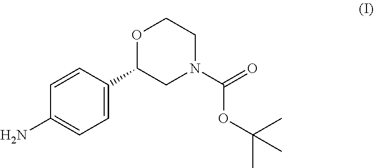 [SMILES: CC(C)(C)OC(=O)N1CCO[C@@H](c2ccc(N)cc2)C1]

(S)-tert-butyl 2-(4-nitrophenyl)morpholine-4-carboxylate (II) (50.0 g), TBME (500 mL, containing <5 ppm water) and water (877 μL) are charged into an autoclave reactor and, under inert conditions, wet 5% Pd/C (Evonik Noblyst® P1093, 582 mg-corresponding to 275 mg on dry basis) is added to the mixture. The reactor is sealed and made inert again by washing the gas phase with argon. The gas phase is washed with hydrogen 5 times and the pressure released. The reactor is heated under stirring to Tᵢ=40±2° C. and then pressurized to 3 barg of hydrogen. Stirring is continued for 6 h while maintaining a constant internal temperature (Tᵢ=40±2° C.) and hydrogen pressure (Pᵢ=3 barg) while recording the hydrogen consumption over time as a measure of the reaction conversion. After 6 h, the temperature is increased to Tᵢ=60±2° C. and the reaction is continued at this temperature for 18 h. After this time, the reactor is cooled to Tᵢ=20±5° C., the stirring stopped, the pressure released carefully and the reactor is made inert with argon. The reaction mixture is filtered to remove the heterogeneous catalyst using a total of 180 mL TBME to wash the reactor and the filter cake. The resulting solution is concentrated to ˜140 mL by distilling the solvent at ambient pressure and then cooled to Tᵢ=50±2° C. n-Heptane (75 mL) is added to the concentrated solution within ≥30 min and the resulting solution is seeded with (S)-tert-butyl 2-(4-aminophenyl)morpholine-4-carboxylate (250 mg as a slurry in 5 mL of n-heptane). The resulting mixture is stirred at Tᵢ=50±2° C. for ≥30 min and then more n-heptane (125 mL) is added within 30 min maintaining Tᵢ=50±2° C. The mixture is cooled under stirring to Tᵢ=20±3° C. within 3 h (cooling ramp: 10° C./h) and stirring is continued at this temperature for ≥12 h. The resulting suspension is filtered and the filter cake washed with a mixture of n-heptane (80 mL) and TBME (40 mL). The wet cake is dried under vacuum at 50±3° C. until constant weight is attained to afford the title compound (40.2 g) as a white solid.

### Example 2

### Preparation of (S)-tert-butyl 2-(4-aminophenyl)morpholine-4-carboxylate (I) in Dry Solvent

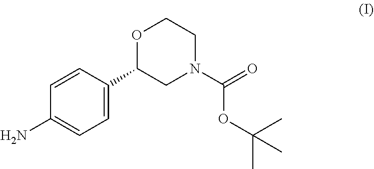 [SMILES: CC(C)(C)OC(=O)N1CCO[C@@H](c2ccc(N)cc2)C1]

(S)-tert-butyl 2-(4-nitrophenyl)morpholine-4-carboxylate (II) (50.0 g) and TBME (100 mL, containing <5 ppm water) are charged in an autoclave reactor and, under inert conditions, wet 5% Pd/C (Evonik Noblyst® P1093, 116 mg-corresponding to 55 mg on dry basis) is added to the mixture. The reactor is sealed and made inert again by washing the gas phase with argon. The gas phase is washed with hydrogen 5 times and the pressure released. The reactor is heated under stirring to Tᵢ=40±2° C. and then pressurized to 3 barg of hydrogen. Stirring is continued for 5 h while maintaining a constant internal temperature (Tᵢ=40±2° C.) and hydrogen pressure (Pᵢ=3 barg) while recording the hydrogen consumption over time as a measure of the reaction conversion. After 5 h, the temperature is increased to Tᵢ=60±2° C. (increasing the temperature determined a faster consumption of hydrogen, that was complete within 6.5 h) and the reaction is continued at this temperature for 18 h. After this time, the reactor is cooled to Tᵢ=20±5° C., the stirring stopped, the pressure released carefully and the reactor is made inert with argon. The reaction mixture is filtered to remove the heterogeneous catalyst using a total of 50 mL TBME to wash the reactor and the filter cake. The resulting solution is concentrated to ˜25 mL by distilling the solvent at ambient pressure and then cooled to Tᵢ=50±2° C. n-Heptane (50 mL) is added to the concentrated solution within ≥1 h. The resulting mixture is cooled under stirring to Tᵢ=0±3° C. within 5 h (cooling ramp: 10° C./h) and stirring is continued at this temperature for ≥12 h. n-Heptane (20 mL) is added at Tᵢ=0±3° C. within 30 min and the mixture is stirred at 0° C. for additional 4 h. The resulting suspension is filtered and the filter cake washed with n-heptane (40 mL). The wet cake is dried under vacuum at 50±3° C. until constant weight is attained to afford the title compound (8.29 g) as a white solid.

1. A process for manufacturing an aniline 2, wherein PG denotes hydrogen or an amino protective group 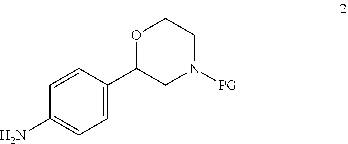
said process comprising:
reacting with hydrogen a nitroarene 1, wherein PG denotes an amino protective group 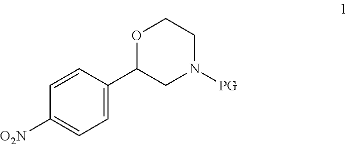
(i) in the presence of a transition metal catalyst;
(ii) in an aprotic solvent;
to form said aniline 2, wherein said aprotic solvent contains 0.01% wt/wt to 0.1% wt/wt of water relative to the aprotic solvent, and wherein the amino protective group is selected from Boc (t-butoxycarbonyl), benzyl, 4-methoxybenzyl, benzhydryl, Fmoc (fluorenylmethoxycarbonyl), Cbz (benzyloxycarbonyl), Moz (p-methoxybenzyl carbonyl), Troc (2,2,2-trichloroethoxycarbonyl), Teoc (2-(Trimethylsilyl)ethoxycarbonyl), Adoc (adamantoxycarbonyl), formyl, acetyl, and cyclobutoxycarbonyl.

2. The process according to claim 1, wherein the amino protective group is Boc (t-butoxycarbonyl).

3. The process according to claim 1, wherein said nitroarene 1 is nitroarene 1a, and wherein said aniline 2 is aniline 2a

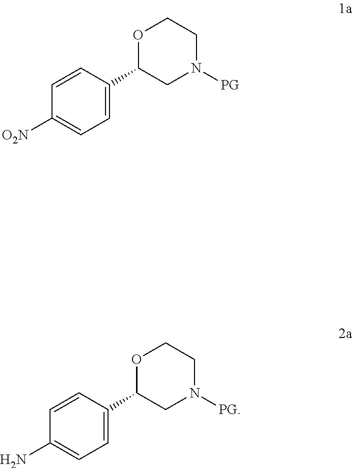

4. The process according to claim 1, wherein said nitroarene 1 is nitroarene 1b, and wherein said aniline 2 is aniline 2b

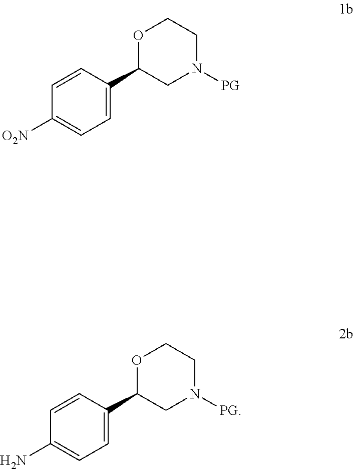

5. The process according to claim 1, wherein said transition metal catalyst is selected from Pt, Pd, Pt—V and Ni, wherein each of said Pt, Pd, Pt—V and Ni is on a solid support.

6. The process according to claim 24, wherein said transition metal catalyst is Pd/C.

7. The process according to claim 6, wherein said Pd/C contains 5% wt/wt of palladium relative to charcoal (5% Pd/C).

8. The process according to claim 1, wherein the catalyst loading is 0.1% wt/wt to 1% wt/wt relative to nitroarene 1.

9. The process according to claim 8, wherein the catalyst loading is 0.4% wt/wt to 0.6% wt/wt.

10. The process according to claim 1, wherein said aprotic solvent is an ether.

11. The process according to claim 10, wherein said ether is tert-butyl methyl ether (TBME).

12. The process according to claim 11, wherein said aprotic solvent contains 0.05% wt/wt to 0.5% wt/wt of water relative to the aprotic solvent.

13. The process according to claim 12, wherein said trace amounts of water are 0.25% wt/wt relative to the aprotic solvent.

14.-15. (canceled)

16. The process according to claim 1, wherein said process is conducted at an elevated temperature of 34° C. to 60° C.

17. (canceled)

18. The process according to claim 1, wherein said process is conducted at an elevated hydrogen pressure of 1 barg to 10 barg.

19. The process according to claim 18, wherein said elevated hydrogen pressure is 3 barg.

20. An aniline 2, wherein PG denotes hydrogen or an amino protective group 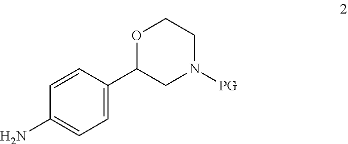
when manufactured according to the process of claim 1, wherein PG denotes an amino protective group selected from Boc (t-butoxycarbonyl), benzyl, 4-methoxybenzyl, benzhydryl, Fmoc (fluorenylmethoxycarbonyl), Cbz (benzyloxycarbonyl), Moz (p-methoxybenzyl carbonyl), Troc (2,2,2-trichloroethoxycarbonyl), Teoc (2-(Trimethylsilyl)ethoxycarbonyl), Adoc (adamantoxycarbonyl), formyl, acetyl, and cyclobutoxycarbonyl

21. A process for manufacturing 5-ethyl-4-methyl-N-[4-[(2S) morpholin-2-yl]phenyl]-1H-pyrazole-3-carboxamide (Formula IV), or a pharmaceutically acceptable salt thereof, 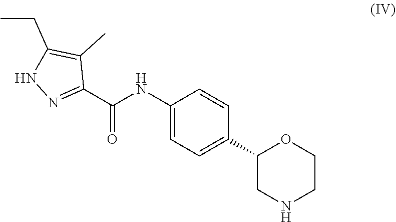 [SMILES: CCc1[nH]nc(C(=O)Nc2ccc([C@H]3CNCCO3)cc2)c1C]
comprising the process according to claim 1.

22.-23. (canceled)

24. The process of claim 5, wherein the transition metal catalyst is selected from PtO2, Pd/C, Pt—V/C, Pt/C, and Raney Ni.

25. The process of claim 9, wherein the catalyst loading is 0.5% wt/wt relative to nitroarene 1.

26. The process of claim 9, wherein the catalyst loading is 0.55% wt/wt relative to nitroarene 1.

27. A process for manufacturing an aniline 2, wherein PG denotes hydrogen or Boc (t-butoxycarbonyl) 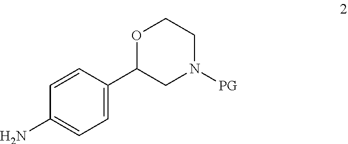
said process comprising:
reacting with hydrogen a nitroarene 1, wherein PG denotes an amino protective group 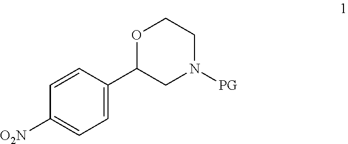
(i) in the presence of a transition metal catalyst;
(ii) in tert-butyl methyl ether (TBME);
to form said aniline 2, wherein said TBME contains 0.01% wt/wt to 0.1% wt/wt of water relative to the TBME.
